(19) United States
(12) Patent Application Publication
(10) Pub. No.: US 2005/0106554 A1
(43) Pub. Date: May 19, 2005

(54) Cryopreservation of pluripotent stem cells

(76) Inventors: Sean P. Palecek, Madison, WI (US); Juan J. de Pablo, Madison, WI (US); Lin Ji, Madison, WI (US); James A. Thomson, Madison, WI (US)

Correspondence Address: QUARLES & BRADY LLP, FIRSTAR PLAZA, ONE SOUTH PINCKNEY STREET, P.O. BOX 2113 SUITE 600, MADISON, WI, 53701-2113, US

(21) Appl. No.: 10/993,468

(22) Filed: Nov. 19, 2004

Related U.S. Application Data
(60) Provisional application No. 60/523,343, filed on Nov. 19, 2003.

(51) Int. Cl. A01N 1/00; C12N 5/08

(52) U.S. Cl. 435/2; 435/366

(57) ABSTRACT

The present invention relates to methods and compositions for the cryopreservation of pluripotent cells in general and human embryonic stem (ES) cells in particular. The stem cells are grown on a bottom layer of solid support matrix and subsequently covered by a top layer of solid support matrix forming a matrix-cell-matrix composition, to which an effective amount of cryopreservation media is added, prior to freezing. The methods of the invention yield cryopreserved cells that exhibit an increase in cell viability and a decrease in cell differentiation, facilitating storage, shipping and handling of embryonic stem cell stocks and lines for research and therapeutics.

### CROSS-REFERENCE TO RELATED APPLICATIONS

This application claims the benefit of U.S. Provisional Application No. 60/523,343 filed Nov. 19, 2003, which is hereby incorporated by reference herein.

### STATEMENT REGARDING FEDERALLY SPONSORED RESEARCH OR DEVELOPMENT

This invention was made with United States government support awarded by the following agency: NAVY/ONR N66001-02-C-8051. The United States has certain rights in this invention.

### BACKGROUND OF THE INVENTION

Over the past several years, significant progress has been made in cryopreservation and lyophilization of biological systems. Most preservation protocols for living cells rely on the addition of dimethyl sulfoxide (DMSO) at concentrations from 5-20% (McLellan, M. R., and Day, J. G. (1995) Methods Mol Biol 38: 1-5). Other chemicals such as glycerol, ethylene glycol, hydroxycellulose, or the disaccharides sucrose, maltose, and trehalose have been shown to enhance cell viability when combined with DMSO (Gulliksson, H. (2000) Transfus Med 10: 257-264). Presumably, these treatments stabilize the cell membrane and/or cell proteins during freezing and drying by forming a glassy material at or near the surface of the cell structure. The ideal protectant interacts favorably with cells and other biological materials, is nontoxic, protects during both freezing and drying, substitutes for water, and has a high glass transition temperature. Based on recent work by researchers, several disaccharides have been found to satisfy these criteria. Trehalose-based formulations appear most promising.

Trehalose is a disaccharide found at high concentrations in a wide variety of organisms that are capable of surviving almost complete dehydration (Crowe et al., (1992) Anhydrobiosis. Annu. Rev. Physiol., 54, 579-599). Trehalose has been shown to stabilize certain cells during freezing and drying (Leslie et al., (1994) Biochim. Biophys. Acta, 1192, 7-13; Beattie et al., (1997) Diabetes, 46, 519-523). Also trehalose-based solutions appear to be remarkably good at forming fragile glasses that protect proteins and cells. In low moisture environments, trehalose is believed to maintain thermodynamic stability of membranes by preserving phospholipid head group spacing and inhibiting lipid phase transitions and separation during freezing and drying. Glassy trehalose matrices slow down kinetic processes in stabilized samples by reducing water mobility and other relaxation processes.

Much of the knowledge in the field of biological preservation originates from work on protein and pharmaceutical stabilization by polymers and disaccharides (Hancock, B. C., and Zografi, G. (1997) J Pharm Sci 86: 1-12; and Miller, D. P., Anderson, R. E., and de Pablo, J. J. (1998) Pharm Res 15: 1215-1221.). Polymers raise the glass transition temperature of the system, and disaccharides preserve protein structure during dehydration or freezing by forming glasses that alter interactions between the protein and water (Miller et al., 1998; and Sano, F., Asakawa, N., Inoue, Y., and Sakurai, M. (1999) Cryobiology 39: 80-87). Recent X-ray crystallography data on lysozyme stabilized with trehalose suggests that the disaccharide does not directly interact with the protein, but instead forms hydrogen bonds with water molecules surrounding the protein, thereby altering the way in which water interacts with the protein (Datta, S., Biswal, B. K., and Vijayan, M. (2001) Acta Crystallogr Biol Crystallogr 57: 1614-1620).

Using molecular simulations of aqueous disaccharide systems, it has been shown that trehalose exhibits a greater ability to hydrogen bond with water than other disaccharides (Ekdawi-Sever, N. C., Conrad, P. B., and de Pablo, J. J. (2001) J Phys Chem 105: 734-742), thereby anticipating and confirming many of the findings that are beginning to emerge from scattering experiments. The simulations of ionic species in disaccharide systems have also allowed the creation of novel formulations containing cross-linked trehalose that have improved considerably the stability of cryopreserved and freeze-dried proteins (Miller, D. P., Anderson, R. E., and de Pablo, J. J. (1998) Pharm Res 15: 1215-1221). These formulations are now being used commercially to stabilize a number of pharmaceutical and biological products, including PCR enzymes.

Furthermore, it has been demonstrated that besides proteins, some of the recent formulations can also increase the viability and long-term stability of single-celled organisms, bacteria and fungi. For example, freeze-dried Lactobacillus acidophilus bacteria was recovered after storage for more than three months at elevated temperatures (Conrad, et al., (2000) Cryobiology 41: 17-24). In fact, many bacteria and fungi, synthesize trehalose from glucose to prevent damage due to extreme temperatures or osmotic shock. Some multicellular organisms, such as the fruit fly Drosophila melanogaster and the plant Arabidopsis thaliana, also synthesize trehalose that protects the organism from a variety of stresses (Leyman, B., et al., (2001) 6: 510-513.)

However, cryopreservation and lyophilization of eukaryotic cells, has posed additional challenges. In contrast to eukaryotic cells, bacteria have evolved stress responses to dehydration and temperature extremes. Also, bacteria possess a cell wall that imparts mechanical stability upon volume changes during freezing or drying, and may shield the cell membrane during ice crystal formation. Eukaryotic cells possess intracellular membranes that increase the number of structures requiring preservation and may provide additional barriers to protectant transport. Thus, additional care must be taken during human cell preservation to maintain cell integrity and viability.

These are serious challenges. However, it has been recently found that intracellular trehalose concentrations on the order of 0.2M allow approximately 75% of human keratinocytes or murine 3T3 fibroblasts to survive a freeze-thaw cycle that kills virtually all nontreated cells (Eroglu, A., et al., (2000) Nat Biotechnol 18: 163-167). Also, it has been reported that the addition of trehalose formulations to cryopreserved human pancreatic islets doubles viable cell recovery and does not affect cell functions upon thawing (Beattie, et al., (1997) Diabetes 46: 519-523). Another study reports that trehalose concentrations of 80 mM increase the survival rate of a mouse fibroblastoid cell line following partial dehydration induced by osmotic shock but could not confer resistance to drying in air (Garcia de Castro, A., and Tunnacliffe, A. (2000) FEBS Lett 487: 199-202).

An alternative approach to cell preservation is vitrification. Vitrification offers promise in enhancing mammalian cell viability following cryopreservation, and can be achieved by combining the use of concentrated protectant solutions with rapid freezing to inhibit ice formation. Vitrification has been extensively used in embryonic preservation, with higher efficiency than other freezing and thawing protocols (Lane, et al., (1999) Nat Biotechnol 17: 1234-1236). Recently, capillary vitrification of human embryonic stem cells in DMSO/ethylene glycol solutions was shown to enhance survival of cryopreserved cells greater than an order of magnitude as compared to slow freezing and fast thawing methods (Reubinoff, et al., (2001) Hum Reprod 16: 2187-2194). However, both slow and rapid methods of freezing can induce background spontaneous differentiation. Thus, protocols need to be optimized to minimize this spontaneous differentiation (Reubinoff et al., 2001).

Preservation of pluripotent stem cells poses additional challenges (Gorlin, J. (1996) J Infus Chemother 6: 23-27). Not only must the cells remain viable, but they must also retain their differentiative capacity (i.e., be maintained in an undifferentiated state). Thus, certain signal transduction pathways must remain in place, and the stresses associated with freezing and drying must not induce premature or erroneous differentiation.

One specific type of pluripotent stem cells, human embryonic stem (HES) cells, are extremely sensitive to the thermal and osmotic stresses experienced during cryopreservation. In fact, less than 0.1% of these cells survive standard cryopreservation of HES cell colonies in DMSO and fetal bovine serum (FBS)-containing medium, primarily due to induction of apoptosis. However, it remains unclear why human ES cells are so sensitive; current hypotheses include differences in membrane compositions, fragile mitotic spindles, fracturing of cell-cell contacts necessary for survival, and slow rates of heat and mass transport through the multicellular colonies. Of the stem cells that do survive cryopreservation, a significant number differentiate shortly following thawing. The premature or erroneous differentiation requires extra time and labor-intensive methods to isolate a pure HES cell population. Thus, improved cell preservation methods and compositions are desired.

### BRIEF SUMMARY OF THE INVENTION

The present invention is summarized as providing methods and compositions for cryopreservation of human pluripotent cells such as embryonic stem (ES) cells. Particularly, to facilitate research studies and clinical applications of stem cells, applicants have developed a novel cryopreservation approach based on stabilizing stem cell colonies adherent to or maintained in a solid support matrix. It has been shown that this method increases cell viability by over an order of magnitude compared to cryopreservation in suspension and reduces differentiation. Applicants have also shown that loading adherent stem cells with the disaccharide trehalose prior to cryopreserving in a dimethyl sulfoxide-containing cryoprotectant solution further improves cell viability under certain conditions.

Accordingly, one embodiment of the invention provides for growing the cells on a bottom layer of solid support matrix, followed by the addition of a top layer of solid support matrix over the cells, such that cells are encapsulated or maintained between the two layers of matrix, forming a matrix-cell-matrix composition. An effective amount of cryopreservation media is then added over the composition, prior to freezing. Upon thawing, the cryopreserved cells exhibit an increase in recovery and viability and a decrease in cell differentiation compared with cells preserved in suspension.

In one aspect, the invention provides that the solid support matrix is either porous, suitably Matrigel™ or non-porous, suitably polystyrene coated with extracellular matrix proteins.

In another aspect, the invention provides that the cells be cultured on a bottom layer of solid support matrix, which serves to anchor the cells after freezing.

In another aspect, the invention provides that a top layer of solid support matrix suitably containing Matrigel™ with conditioned medium be poured over the cells to prevent cell detachment prior to freezing.

In another aspect, the invention provides for use of cryopreservation medium to be added over the top matrix; wherein the cryopreservation media optionally, includes the addition of a carbohydrate-based medium, followed by addition of a freezing medium immediately prior to cooling the cells; wherein the cryopreservation media is capable of supporting growth and inhibiting differentiation.

In this aspect of the invention the carbohydrate in the carbohydrate based medium is a disaccharide, preferably trehalose.

In another aspect, the invention provides that the freezing medium contains 10% DMSO, 30% FBS and 60% HES medium.

In one aspect, the invention provides that the embryonic stem cells be mammalian cells, and suitably human cells.

In another embodiment, the invention provides for a matrix-cell-matrix composition, which includes a bottom layer of solid support matrix with conditioned medium; embryonic stem cells grown on the bottom layer; and a thin top layer of solid support matrix in conditioned medium poured over the cells, such that cells are encapsulated or maintained between the two layers of matrix, forming a sandwich culture composition.

One advantage of the invention is that it provides a reduction in the time required to amplify frozen stocks of embryonic stem cells, and minimizes the risk of clonal selection during freeze-thaw cycles.

Another advantage of the invention is that it facilitates storage, shipping and handling of cryopreserved embryonic stem cell stocks, lines and cell clone libraries for use in research and clinical settings.

In another embodiment, the invention provides a method of cryopreserving embryonic stem cells, by growing the cells on a bottom layer of solid support matrix, such that the cells adhere to the matrix. An effective amount of a cryopreservation media is poured over the matrix adherent cells; wherein the cryopreservation media is capable of supporting growth and inhibiting differentiation. The cells are then cooled to a temperature sufficient to cryopreserve them.

In another embodiment, the invention provides for a matrix-cell-matrix composition, wherein the bottom matrix includes matrix-coated beads, such as Cytodex microcarriers, on which embryonic cells are grown. A top layer of matrix, such as Matrigel™ is added over the cells encapsulating the cells between the matrices. This composition is then cryopreserved using the methods described herein.

In yet another embodiment, the invention provides for a matrix-cell composition, wherein the bottom matrix includes matrix-coated beads, such as Cytodex microcarriers, on which embryonic cells are grown. This composition is then cryopreserved using the methods described herein.

Unless otherwise defined, all technical and scientific terms used herein have the same meaning as commonly understood by one of ordinary skill in the art to which this invention belongs. Although suitable methods and materials for the practice or testing of the present invention are described below, other methods and materials similar or equivalent to those described herein, which are well known in the art, can also be used.

Other objects, advantages and features of the present invention will become apparent from the following specification taken in conjunction with the accompanying drawings.

### BRIEF DESCRIPTION OF THE SEVERAL VIEWS OF THE DRAWINGS

FIG. 1 graphically illustrates DMSO protects HES cells from membrane rupture during cryopreservation.

FIG. 2 graphically illustrates few HES colonies cryopreserved in suspension attach and grow when replated on Matrigel™.

FIG. 3 graphically illustrates HES colonies cryopreserved adherent to a Matrigel™ substrate exhibit higher viability upon thawing than HES colonies frozen in suspension.

FIG. 4 graphically illustrates cryopreservation of adherent HES colonies significantly increases colony recovery rate compared to cryopreservation of colonies in suspension.

FIGS. 5A-C are a series of photomicrographs showing cryopreservation of adherent HES colonies reduces differentiation upon thawing.

FIGS. 6A-D are a series of photomicrographs showing OCT4 expression in HES cells frozen adherent to and embedded in Matrigel™.

FIG. 7 illustrates karyotype of HES cells frozen embedded in Matrigel™.

FIGS. 8A-D show endocytosis can load lucifer yellow into HES cells.

FIG. 9 graphically illustrates that loading HES cells with trehalose can improve recovery of adherent HES cells in certain freezing medium.

FIG. 10 illustrates that virtually all HES colonies cryopreserved adherent to Matrigel™ were recovered and growing post-thawing.

FIG. 11 illustrates the expression of SSEA4 in cryopreserved and recovered HES cells.

FIG. 12 is a photomicrograph of ES cells grown on a monolayer of laminin-coated Cytodex 3 microcarriers.

### DETAILED DESCRIPTION OF THE INVENTION

The present invention provides methods and compositions for cryopreservation of pluripotent stem cells. The stem cells are grown on a bottom layer of solid support matrix and subsequently covered by a top layer of solid support matrix forming a matrix-cell-matrix composition, over which an effective amount of cryopreservation media is added, prior to freezing. Upon thawing, the cells cryopreserved using the matrix-cell-matrix composition exhibit an increase in cell viability and a decrease in cell differentiation. These methods enable a decrease in the time required to recover preserved cells from 4-6 weeks down to only several days and may facilitate storage, shipping and handling of stem cell stocks and cell lines. Thus, the methods of the present invention provide enormous medical potential in areas such as human developmental biology and cell-based therapies.

In the broadest sense the methods of the invention relate to cryopreservation of pluripotent stem cells in general, and embryonic stem (ES) cells in particular. The stem cells were grown in culture on a bottom layer of solid support matrix with medium on either a standard hard polystyrene surface or a flexible surface, such as Bioflex® culture plates. As used herein the term “medium” or “media” refers to a cell culture solution that is capable of supporting growth and inhibiting differentiation. The ES cells may be cultured in conditioned or unconditioned media as described below in the examples. A medium is referred to as conditioned when it has been previously used to culture fibroblast cells, a treatment which confers upon the medium the trait of sufficiency to culture stem cells in an undifferentiated state without feeder cells. Other media, as described below, will support stem cells in an undifferentiated state, without the need for conditioning or feeder cells. As used herein the term “cells” encompasses pluripotent cells and specifically includes embryonic stem cells. To prepare the cell culture for freezing, the culture was processed by adding a top layer of solid support matrix with medium over the cells, such that cells were effectively maintained between the two layers of matrix forming a matrix-cell-matrix composition. It is noted that cells begin to die during the detachment process from the matrix. By helping to keep the cells attached to the matrix during the freezing process by adding a top matrix layer, not only is the viability of the thawed cells maximized, but the cells are also maintained in an undifferentiated state.

As used herein the phrase “solid support matrix” refers to either a porous or non-porous solid support matrix that facilitates cell growth and inhibits differentiation. For example, a preferable extracellular porous matrix is Matrigel™ in conditioned medium. It is envisioned that other less expensive alternatives to Matrigel™ may be used in practicing the methods of the invention. These non-limiting alternatives may include collagen, hyaluronic acid, gelatin material, Elastin, ProNectin and Laminin or mixtures thereof to anchor the cells to the matrix after freezing. Also, for example, a suitable non-porous matrix that may be used to support cell growth is polystyrene coated with extracellular matrix (ECM) proteins or non-porous beads coated with ECM proteins (e.g., laminin.)

Although it would be preferable to have Matrigel™ with conditioned medium as both the bottom and top layer of the matrix-cell-matrix composition, it is envisioned that the matrix layers could be made of different material. For example, the cells may be grown on Mouse Embryonic Fibroblast feeder cells (MEFs), permitting continuous undifferentiated growth and obviating the need for conditioned medium. A top layer of Matrigel™ may be poured over the cells to keep the cells attached to the bottom matrix layer during the freezing process, yielding maximum viability of thawed cells while maintaining the cells in an undifferentiated state.

Furthermore, in referring to the solid support matrix, it is envisioned that the two matrix layers may be either of equivalent or different thickness. However, applicants believe that it maybe better in terms of nutrient transport if the top layer is thinner. Therefore, preferably, the top matrix layer may be thinner than the bottom layer of matrix on which the cells are cultured. Alternatively, in experiments performed where the top layer is equivalent to or thicker than the bottom layer no significant difference in results was observed.

After a top layer of matrix was added to the cells, then an effective amount of cryopreservation media was added over the matrix-cell-matrix composition. As used herein the phrase “cryopreservation media” refers to media containing cryopreservatives which include, but are not limited to carbohydrates, DMSO, and FBS, in a medium, which is capable of supporting growth and inhibiting differentiation. Most suitably, in the present method two different types of cryopreservation media were utilized: a “carbohydrate-based conditioned medium” also referred to herein as “trehalose loading medium” followed by a “freezing medium.” As used herein the phrase “carbohydrate-based conditioned medium” refers to a medium containing an effective amount of carbohydrate, preferably, a disaccharide, such as trehalose in medium, preferably in conditioned medium.

The disaccharide trehalose is a cryoprotectant/lyoprotectant that has demonstrated effectiveness in protecting mammalian cells during freezing and drying. Trehalose in stem cell conditioned medium has been found by the applicants to help stabilize and preserve proteins during freezing, freeze-drying, and air-drying. Trehalose is theorized to protect cells from freezing and freeze-drying through one or more of the following mechanisms: counterbalancing external osmotic pressure, stabilizing biomolecules via preferential exclusion, forming a protective glass around biological molecules, and preventing damaging phase transitions in lipid membranes (Crowe, J. H, et al., (1998) Annu Rev Physiol 60: 73-103). In accordance with the invention, trehalose is believed to associate with the head groups of phospholipids and maintain the phospholipid spacing in the cell membrane as water is removed. However, one of the disadvantages of trehalose is that it does not easily penetrate lipid bilayers, and must be loaded into cells through endocytosis or other methods that temporarily disrupt the cell membrane.

Also, as used herein the phrase “freezing medium” refers to a medium containing an effective amount of FBS, DMSO and HES medium to facilitate cryopreservation of the cells. Typically, the freezing medium composition includes between 5-15% by volume of DMSO and serum concentrations can range from 20-95%. Most preferably, the freezing medium added to the matrix-cell-matrix includes 10% DMSO, 30% FBS and 60% conditioned HES medium.

After the addition of the carbohydrate-based conditioned media and the freezing medium over the matrix-cell-matrix composition, the culture plate was then suitably wrapped, cooled and stored. For example, the edge of the plate containing the matrix-cell-matrix composition was first sealed with parafilm, wrapped with a layer of saran wrap and covered with several layers of paper towels. The plate was put into a styrofoam box and placed into a −80° C. freezer. It is encompassed that the cooling temperatures maybe anywhere from −70° C. to −200° C. The box was then stored in liquid nitrogen. It is also contemplated that the matrix-cell-matrix composition could be preserved by freeze-drying (lyophilization), a two-step process in which the sample is first frozen and then dried at low temperature under vacuum.

As an aside, typically, upon cooling, as the external media freezes, cells equilibrate by losing water, thus increasing intracellular solute concentration. Below about 10 to 15° C. intracellular freezing will occur. Both intracellular freezing and solution effects are responsible for cell injury (Mazur, P., (1970) Science 168:939-949).

It has been proposed that freezing destruction from extracellular ice is essentially a plasma membrane injury resulting from osmotic dehydration of the cell (Meryman, H. T., et al., (1977) Cryobiology 14:287-302). Also, different optimal cooling rates have been described for different cells. Various groups have looked at the effect of cooling velocity or cryopreservatives upon the survival or transplantation efficiency of frozen cells (Mazur, P., (1970) Science 168:939-949. Substantial time and effort has been expended in an effort to develop cryoprotective agents and establish optimal cooling rates without achieving an improved increase in the viability, much less maintaining the thawed cells in an undifferentiated state.

In accordance with the present invention, after the cells have been stored for the desired period of time, the plate was taken out of the box and the paper towels were removed. The plate was placed in a 37° C. waterbath and thawed as rapidly as possible. After thawing, fresh conditioned medium was added over the top layer of the matrix and the plate was incubated at 37° C. The media may be changed daily and the cells passaged when colony size becomes greater than 10,000 cells. Thus, the above-described process of the invention provides cells having enhanced cell viability and decreased differentiation, as compared to cells not cryopreserved using the above-described method.

It is further noted that in general, the role of thawing temperature on cryopreserved stem cells has not been systematically studied. The prevailing protocol involves thawing the cells at or near their growth temperature, 37° C. for mammalian cells. However, thawing at a lower temperature or slower rate may reduce certain types of damage, such as oxidative stress detected by adhesion-mediated signaling, while permitting membranes to seal any pores formed by ice crystallization. It is believed that adhesion signals play a role during freezing and thawing of stem cells. Furthermore, it is noted that of the matrices screened for promoting stem cell viability, Matrigel™ provides superior resistance to damage during cryopreservation, since the cells receive survival and proliferation signals during both the freezing and thawing components of the process.

Furthermore, it is envisioned that the method of the invention may be used for cryopreservation, recovery and therapeutic use of embryonic stem cells. As desired, the viable and undifferentiated thawed cells could be introduced into a subject in need of such cells. As used herein the term “viability” or “viable” refers to a cell that is capable of normal growth and development after having been cryopreserved and thawed. In the present invention, it is encompassed that viability of the cells maybe determined by a number of methods, well known in the art, such as for example, the MTT assay or the Alamar Blue Assay both of which are described in the examples below.

Also, as used herein the term “differentiation” or “differentiate” refers to a process during which young, immature, embryonic (unspecialized) cells take on individual characteristics and reach their mature (specialized) form and function. Techniques for isolating stable (undifferentiated) cultures of human embryonic stem cells have recently been described by Thomson et al., in U.S. Pat. No. 5,843,780 and J. Thomson et al., 282 Science 1145-1147 (1998), incorporated by reference as if fully set forth below. Stem cell differentiation may be measured by a variety of methods well known in the art, such as for example, by monitoring the presence of stem cell surface markers OCT4 and SSEA-4 using immunofluorescence microscopy, described in the examples below.

Accordingly, a preferred method of the invention provides for growing the cells on a bottom layer of solid porous matrix, followed by the addition of a thin top layer of solid porous matrix over the cells, such that cells are encapsulated or maintained between the two layers of matrix, forming a matrix-cell-matrix composition. An effective amount of a cryopreservation medium is then added over the composition, prior to freezing. Specifically, the carbohydrate based conditioned medium which has between about 20-50 mM trehalose and preferably 35 mM trehalose is poured over the composition. This is done typically between 1 to 30 hours, preferably 24 hours before the freezing medium is added. After a period of time, such as 24 hours, the composition is then frozen. Upon thawing, the cryopreserved cells exhibit an increase in recovery, viability and a decrease in cell differentiation compared with cells preserved in suspension. More specifically, applicants have observed that although the effect of trehalose may be minor compared to the effect of freezing in an adherent state, there is a statistically significant benefit. Accordingly, a preferred method of practicing the invention is to use trehalose in the manner described herein to maximize viability of the cells. Although, it is envisioned that in some applications, the addition of trehalose may be optional and not worth the effort to obtain a statistically significant benefit of improved viability which is ultimately achieved.

Likewise, a preferred composition of the invention includes a matrix-cell-matrix composition, which includes a bottom layer of solid porous matrix with conditioned medium; embryonic stem cells grown on the bottom layer; and a thin top layer of solid porous matrix in conditioned medium poured over the cells. In this embodiment the cells are encapsulated or maintained between the two layers of matrix, forming a sandwich culture composition.

In this embodiment it is encompassed that the bottom layer of solid porous matrix includes matrix-coated beads, suitably nonporous beads coated with laminin or Matrigel™, such as Cytodex™ microcarriers, on which embryonic cells are grown. A top porous or non-porous layer of matrix, preferably, Matrigel™ may be added over the cells encapsulating the cells between the matrices. This composition is then cryopreserved using the methods described below.

In another embodiment, the invention provides a method of cryopreserving embryonic stem cells, by growing the cells on a bottom layer of solid support matrix, such that the cells adhere to the bottom matrix. In this embodiment an effective amount of cryopreservation media is then added over the matrix adherent cells, prior to freezing. Specifically, the carbohydrate based conditioned medium which has between about 20-50 mM trehalose and preferably 35 mM trehalose is poured over the adherent cells. This is done typically between 1 to 30 hours, preferably 24 hours before the freezing medium is added. The cells are then cooled to a temperature sufficient for cryopreservation. Alternatively, a similar result may be obtained without the addition of carbohydrate based conditioned medium. Instead, in this embodiment, the freezing medium is preferably added to the cell-matrix composition for a time period of about 24 hours prior to freezing.

In yet another embodiment, the invention provides for a matrix-cell composition, wherein the bottom matrix includes matrix-coated beads, such as Cytodex™ microcarriers, on which embryonic cells are grown and cryopreserved as described herein the examples.

In practicing the methods of the invention, it is envisioned that the cryopreservation process may have an effect on a variety of cellular processes. The freezing process itself may virtually halt intracellular reactions, including gene transcription. This may result from chemical composition of the protectant formulation, such as metabolic effects of trehalose, high anion concentrations, or low-moisture environment, among others properties. Also, in cryopreservation, the stresses induced by freezing affect cellular transport processes involving heat shock or membrane destabilization proteins.

Another resultant aspect of practicing the method of the invention is that long-term changes in gene expression may follow cryopreservation signifying permanent cellular alterations. If these changes affect the differentiation state of the stem cells or their capacity for unlimited proliferation, their utility would diminish. As such, it is encompassed by the invention that expression changes for differentiation markers or senescence genes (telomerases, etc.) may be analyzed and changes in differentiation state or proliferative capacity may be confirmed using other methods (e.g. antibody binding or telomere length assays) or functional assays for proliferation rate and senescence.

Furthermore, it is envisioned that the cryopreservation advance described by the invention should make it possible to partially lyophilize such matrices to facilitate storage conditions and increase long-term viability and further the ability to store stem cells without refrigeration. This is particularly important for remote locations and to enable centralized stockpiling and easy transport. Thus, the methods of the invention may help to develop effective approaches for lyophilization and rehydration of stem cells to further improve cell recovery and viability with reduced differentiation.

The invention will be further described in the following examples, which do not limit the scope of the invention defined by the claims.

### EXAMPLES

### Materials and Methods

### Example 1

Cell lines and Preparing Feeder Cells.

The HES cell lines H1 and H9 were derived from the inner cell mass of blastocyst stage embryos (Thomson et al. 1998). HES cells were cultured as undifferentiated cells using HES medium, which is capable of supporting growth and inhibiting differentiation and MEF feeder cells or CM/F+medium on Matrigel™-coated plates. HES cells were used between passage number 26 and 40. MEF cells were isolated as described (Thomson et al. 1998) and used between passage 1 and 4. MEF feeder cells were prepared by coating a tissue culture plate with 0.1% gelatin solution, 2 ml/well to a 6-well plate, and 0.5 ml/well to a 24-well plate. After coating, the plate was incubated overnight in a 37° C., humidified incubator with 5% CO₂ for 24 hours prior to plating irradiated MEF cells. 2×10⁵ irradiated MEF cells were added to 2.5 ml MEF medium (90% DMEM, 10% FBS, and 1% MEM non-essential amino acids solution) in each well of a 6-well plate. MEF cells were incubated overnight at 37° C. prior to plating HES cells. All media components were obtained from Invitrogen Corp. and other chemical reagents from Sigma-Aldrich Co.

### Example 2

Preparing Matrigel™ Plate.

To prepare a Matrigel™ plate, a tube of Matrigel™ stock (2 mg) was taken directly from the −20° C. freezer. Matrigel™ was obtained from Becton Dickinson, San Jose, Calif. The Matrigel™ pellet was immediately resuspended in 6 ml ice cold DMEM/F12. All chunks in the mixture were eliminated through vigorous pipetting. A 1 ml aliquot of the Matrigel™ mixture was added to each well of the 6-well plate. The plate was maintained at room temperature for one hour or overnight at 4° C. before use.

### Example 3

Preparing Conditioned Media (CM).

MEF conditioned media (CM) was prepared by coating a T75 flask with 10 mL 0.1% gelatin solution and incubating for 24 hr in a 37° C. humidified incubator with 5% CO₂ prior to plating irradiated (35 Gy γ radiation) MEF cells. Irradiated MEF cells (3×10⁶) were added to 15 ml MEF medium in the T75 flask and incubated overnight at 37° C. The MEF medium was aspirated and discarded. HES medium (20 ml) without bFGF (80% DMEM/F12 medium, 20% Knockout Serum Replacement, 1% L-glutamine solution, and 0.1 mM MEM non-essential amino acids solution) was added to the MEF cells and incubated overnight. The CM was then collected and replaced with 20 ml HES medium without bFGF. CM was collected daily for up to 2 weeks. bFGF was added to the CM to a final concentration of 4 ng/ml to make CM/F+.

### Example 4

Preparing Unconditioned Media (UM)

In accordance with the invention, ES cells lines may also be suitably cultured in media having higher concentrations of FGF but in the absence of both serum and feeder cells. Three different medium formulations are referred to below: UM100, BM+ and DHEM. The nomenclature UM100 refers to unconditioned medium to which has been added 100 ng/ml of bFGF. The UM100 medium does contain the Gibco Knockout Serum Replacer product but does not include or require the use of fibroblast feeder cells of any kind. The BM+ medium is basal medium (DMEM/F12) plus additives, described below, that also permits the culture of cells without feeder cells, but this medium omits the serum replacer product. Lastly, the name DHEM refers to a defined human embryonic stem cell medium. This medium, also described below, is sufficient for the culture, cloning and indefinite proliferation of human ES cells while being composed entirely of inorganic constituents and only human proteins, as opposed to the BM+ medium which contains bovine albumin.

Accordingly, UM100 media may be prepared as follows: unconditioned media (UM) consisted of 80% (v/v) DMEM/F12 (Gibco/Invitrogen) and 20% (v/v) Knockout-Serum Replacer (Gibco/Invitrogen) supplemented with 1 mM glutamine (Gibco/Invitrogen), 0.1 mM β-mercaptoethanol (Sigma-St. Louis, Mo.), and 1% nonessential amino acid stock (Gibco/Invitrogen). To complete the media preferably 100 ng/ml bFGF was added and the medium was filtered through a 0.22 uM nylon filter (Nalgene). However, bFGF between the range 0.1 ng/ml to 500 ng/ml is suitable.

BM+ medium was prepared as follows: 16.5 mg/ml BSA (Sigma), 196 μg/ml Insulin (Sigma), 108 μg/ml Transferrin (Sigma), 100 ng/ml bFGF, 1 mM glutamine (Gibco/Invitrogen), 0.1 mM β-mercaptoethanol (Sigma), and 1% nonessential amino acid stock (Gibco/Invitrogen) were combined in DMEM/F12 (Gibco/Invitrogen) and the osmolality was adjusted to 340 mOsm with 5M NaCl. The medium was then filtered through a 0.22 uM nylon filter (Nalgene).

DHEM media was prepared as follows: 16.5 mg/ml HSA (Sigma), 196 μg/ml Insulin (Sigma), 108 μg/ml Transferrin (Sigma), 100 ng/ml bFGF, 1 mM glutamine (Gibco/Invitrogen), 0.1 mM β-mercaptoethanol (Sigma), 1% nonessential amino acid stock (Gibco/Invitrogen), vitamin supplements (Sigma), trace minerals (Cell-gro®), and 0.014 mg/L to 0.07 mg/L selenium (Sigma), were combined in DMEM/F12 (Gibco/Invitrogen) and the osmolarity was adjusted to 340 mOsm with 5M NaCl. It is noted that the vitamin supplements in the media may include thiamine (6.6 g/L), reduced glutathione (2 mg/L) and ascorbic acid PO₄. Also, the trace minerals used in the media are a combination of Trace Elements B (Cell-gro®, Cat #: MT 99-175-Cl and C (Cell-gro®, Cat #: MT 99-176-Cl); each of which is sold as a 1,000× solution. It is well known in the art that Trace Elements B and C contain the same composition as Cleveland's Trace Element I and II, respectively. (See Cleveland, W. L., Wood, I. Erlanger, B. F., J. Imm. Methods 56: 221-234, 1983.) The medium was then filtered through a 0.22 uM nylon filter (Nalgene). Finally, sterile, defined lipids (Gibco/Invitrogen) were added to complete the medium.

### Example 5

Splitting Human Embryonic Stem Cell Culture

To detach the HES colonies from MEF feeder layers or from Matrigel™-coated plates during passage, the medium from HES cell culture plate was aspirated. Collagenase splitting medium (1 ml at 1 mg/ml in DMEM/F12) was added to each well in a 6-well plate. The plate was incubated in a 37° C., humidified incubator with 5% CO₂, for about 3-5 min. It was confirmed that the edges of the colonies were separating from the surface of the plate by microscope inspection. The tip of a glass 5 ml pipet was used to scrape the colonies off the surface of the plate for a 6 well plate. The colony suspension was transferred into a sterile 15 ml conical tube. The cells were gently pipetted up and down a few times in the tube, to further break up the colonies. The cells were pelleted by centrifugation at 1000 rpm for 5 min and the supernatant was aspirated. The cell pellet was washed by adding about 3 ml of HES medium to the 15 ml conical tube and the pellet was gently reconstituted in the HES medium. The mixture was then centrifuged at 1000 rpm for 5 min. While the HES cells were spinning for the second time, the MEF medium was aspirated away from the fresh feeder plate.

The plate was washed once with 2 ml of 1× calcium and magnesium-free PBS solution per well. The supernatant was aspirated from the HES cell pellet after the second spin. A sufficient volume of medium was added and mixed thoroughly to form the desired number of cells for the split. The PBS solution was aspirated from the wells. The HES cells were evenly dispensed among the desired number of wells by adding them dropwise to each well. The HES medium (80% DMEM/F12 media, 20% Knockout Serum Replacer, 1% L-glutamine solution, 0.1 mM MEM non-essential amino acids solution, and 4 ng/ml bFGF) was used on the feeder plate. Alternatively, the conditioned HES medium was used on the Matrigel™ plate. After plating the HES cells, they were returned to the incubator and the plate was moved in several quick, short, back-and-forth and side-to-side motions. The cells were incubated in a humidified 37° C. incubator with 5% CO₂. The culture was refreshed once per day with media. The culture was split about 1:3 to 1:6 ratio, approximately every 5-7 days.

### Example 6

Cryopreserving ES Cells in Suspension

In order to cryopreserve ES cells in suspension, cells were grown to approximately 1000-10,000 cell colonies on Matrigel™ or a MEF feeder layer. Average colony size was determined by counting colony number in a representative sample, then dispersing cells in the colony by treatment with 10 mg/ml dispase and counting cell number. 1 mg/ml collagenase was used to detach colonies from the plate and the colonies were resuspended in freezing medium containing FBS, DMSO and CM at varying concentrations. Cells were not dispersed during freezing. The solution was not seeded to form extracellular ice. 1 ml of 1-3×10⁵ cells/ml were placed in a cryovial and frozen at approximately −1° C. per minute. Initial studies were performed by placing cells in a Nalgene Cryo 1° C. freeze container in a −80° C. freezer, and later samples were frozen in a controlled-rate freezer (Forma Scientific, Model 8026). Differences in freezing method did not significantly affect viability. After reaching −80° C., cells were stored in liquid nitrogen.

After 5-7 days in liquid nitrogen, cryovials were thawed rapidly in a 37° C. water bath and the liquid transferred to a 15 ml tube. HES or CM/F+medium was added in a drop-wise manner and cells were pelleted and resuspended in HES or CM/F+ medium. Cells were then plated on a MEF feeder monolayer or a Matrigel™-coated plate.

### Example 7

Cryopreserving Matrix Adherent ES cells.

In this example, cells were grown to approximately 1000-10,000 cell colonies on Matrigel™ or a MEF feeder layer in a 24 well plate. Average colony size was determined by counting colony number in a representative sample, then dispersing cells in the colony by treatment with 10 mg/ml dispase and counting cell number. The growth medium was aspirated from each well and a top layer of Matrigel™ was poured over the adherent colonies by diluting 6 mg Matrigel™ in 12 ml CM/F+ (conditioned medium+bFGF) and adding 0.5 ml to each well of the 24 well plate. The plate was incubated at 37° C. in a humidified 5% CO₂ incubator for 1 hr and the Matrigel™ solution was aspirated from the plate. Then, 0.5 ml growth medium (HES or CM/F+ was added to each well and cells were cultured in the Matrigel™ for up to 2 days. Growth medium was aspirated from each well and 0.5 ml freezing medium containing varying concentrations of FBS, DMSO, and CM was added to each well. The fresh freezing medium was made on the day of freezing. The freezing medium generally contains 10% DMSO, 30% FBS and 60% conditioned HES media. The medium on the plate was aspirated off and replaced with 0.5 ml freezing medium.

The plate was sealed with parafilm and frozen to −80° C. at −1° C. per minute. Initial studies were performed by placing cells in a parafilm-sealed Styrofoam box in a −80° C. freezer, and later samples were frozen in a controlled-rate freezer (Model 8026, Forma Scientific). Differences in freezing method did not significantly affect viability. The solution was not seeded to form extracellular ice. The plates containing the cells were then transferred to liquid nitrogen and stored 5-7 days, which was long enough to stabilize the cells but permitted a reasonable experimental throughput.

### Example 8

Thawing HES Cells.

To thaw the ES cells, plates were placed in a 37° C. water bath and swirled to thaw as rapidly as possible. After thawing, 1 ml fresh CM/F+ medium was added in a drop-wise manner to each well, then aspirated. Fresh media (0.5 ml) was added to each well and the cells were incubated at 37° C. in a humidified 5% CO2 incubator. Media was changed daily and cells passaged by using 1 ml of 1 mg/ml collagenase to remove cells from Matrigel™ encapsulation or a Matrigel™-coated surface. Cells were transferred to fresh CM/F+ medium in a new Matrigel™-coated T75 flask or well in a multiwell plate.

### Example 9

Loading Trehalose Into Matrix Adherent HES Cells.

Trehalose loading medium was prepared by dissolving trehalose in CM/F+ to a concentration of 35 mM. Cells were incubated in trehalose loading medium for up to 2 days prior to transfer to freezing medium.

### Example 10

Techniques Used for Measuring Cell Viability.

In order to determine the level of cell viability, applicants used a) trypan blue staining; b) MTT assays; and c) alamar blue assays as described below; however other methods known to those skilled in the art may be used for cell viability measurements.

A. Trypan Blue Staining

HES cells were harvested from a flat-bottomed 24-well tissue culture plate coated with Matrigel™ by addition of trypsin-EDTA (0.25% trypsin, 1 mM EDTA) until colonies were completely dispersed. HES cells were resuspended in 500 μl HES medium containing 10% FBS. HES cell suspension (10 μl of the) was added to 10 μl 4% Trypan blue solution and 80 μl PBS. Total cell number and the fraction of cells that incorporated Trypan blue were counted on a hemacytometer.

B. MTT Assay

HES cells were cultured grown on flat-bottomed 24-well tissue culture plates coated with Matrigel™, with 0.5 ml CM/F+ in each well. 0.05 ml MTT solution (3-[4,5-dimethylthiazol-2-yl]-2,5-diphenyltetrazolium bromide) was added to each well. Wells were mixed by tapping gently on the side of the plate and incubated at 37° C. for 2 to 4 hours for reaction of MTT to occur. Then 0.5 ml isopropanol containing 0.04 N HCl was added to each well to stop MTT reaction. Wells were mixed thoroughly by repeated pipetting. Within an hour, absorbance at 595 nm was measured on a plate reading spectrophotometer (TECAN Genios). A standard curve for the MTT assay (not shown) was generated by growing HES colonies to different concentrations, ranging from 5×10⁴ cells per well to 2×10⁶ cells per well (determined by counting dispersed cells on a hemacytometer) on a Matrigel™-coated 24 well plate.

C. Alamar Blue Assay

HES cells were cultured on flat-bottomed 24-well tissue culture plates coated with Matrigel™, with 0.5 ml CM/F+ in each well. 0.05 ml Alamar blue solution was added to each well. Wells were mixed by tapping gently on the side of the plate and incubated at 37° C. for 3 hours. Then absorbance was measured on a TECAN Genios plate reader at 595 nm. A standard curve for the Alamar blue assay (not shown) was generated by growing HES colonies to different concentrations, ranging from 5×10⁴ cells per well to 2×10⁶ cells per well (determined by counting dispersed cells on a hemacytometer) on a Matrigel™-coated 24 well plate.

### Example 11

Techniques Used for Evaluating Cell Differentiation

While cell viability is a critical determinant of cryoprotectant function, it is not the only important parameter. For ES cells to be applicable for a variety of medical applications, the cells must retain the capacity for unlimited proliferation and differentiation. Any differentiation will limit their use in downstream applications. To measure differentiation in stem cells, the level of surface markers such as OCT4 and SSEA-4 is monitored by immunofluorescence microscopy (Xu, C., et al., (2001) Nat Biotechnol 19: 971-974, incorporated by reference herein in its entirety). Specifically, OCT4 is an embryonic gene transcription factor that plays an role in control of developmental pluripotency, so that when OCT4 gene activity is repressed in pluripotent stem cells differentiation occurs. (See, Pesce, et al., (1998) Mech. Dev., 71:89).

In general, cell morphology was viewed and imaged using an inverted culture microscope. HES cells were best viewed at a lower objective, such as 1.6×, where several colonies could be observed at once, as well as at a higher objective, such as 10× or 20×, where individual colonies and cell morphology can be observed.

A. Immunocytochemistry

OCT4 expression of cryopreserved HES cells was determined by immunocytochemistry. After thawing, cells were washed in PBS and fixed in 3.7% paraformaldehyde for 1 hr at 4° C. Cells were then permeabilized with 0.2% Triton X-100 for 1 hr at room temperature and washed three times in PBS. Samples were incubated with the primary anti-OCT4 antibody (Santa Cruz) for 1 hr at room temperature at 1:100 concentration followed by washing. A secondary antibody, fluorescein-labeled goat anti-rabbit IgG, was applied for 1 hr at room temperature at 1:1000 concentration, followed by washing 3 times in PBS. Samples were imaged using phase contrast and immunofluorescent microscopy.

B. Flow Cytometry

SSEA4 expression was determined by flow cytometry. After thawing, cells were allowed to grow on Matrigel™ in CM/F+ medium for 7 days, then colonies were removed from Matrigel™ by 1 mg/ml collagenase IV (GIBCO/BRL) treatment. Cells were dispersed by treatment with a 0.05% trypsin, 0.53 mM EDTA solution (GIBCO/BRL) for 5-10 minutes and filtered through a 40 μm mesh. 100 μl of the cell suspension containing 5×10⁶ cells/ml was added to a sample tube and a control tube. 1 μl of MC813-70 (anti-SSEA4) (Kannagi et al. 1983) was added to the test tube and 5 μl of 1 mg/ml mouse IgG (Sigma) was added to the control tube. Tubes were vortexed and incubated on ice for 30 min. 1 μl flourescein-labeled rabbit anti-mouse IgG (Pierce) was then added and tubes were incubated for another 30 minutes on ice. Cells were washed three times and suspended in 0.3 ml of calcium and magnesium free PBS+2% FBS+0.1% sodium azide+5 pg/ml propidium iodide. Samples were analyzed using a FACScan flow cytometer (Becton Dickinson) and Cellquest software (Becton Dickinson). At least 10,000 events were acquired for each sample and analysis was restricted to intact cells based on light scatter properties, as well as propidium iodide exclusion to remove dead cells from analysis. The fluorescein signal was obtained through a 530/30 band pass filter and the mean fluorescence values for the IgG control and test samples were determined.

C. Karyotypying

HES colonies were harvested from a MEF monolayer, then transferred to a Matrigel™-coated 24 well plate. Colonies were cultured then treated with an additional layer of Matrigel™ 24 hours prior to freezing. Freezing and thawing was performed as described in Cryopreservation of Adherent Cells. Cells were maintained in liquid nitrogen for 7 days prior to thawing. Following thawing, colonies were harvested by treatment with 1 mg/ml collagenase and transferred to HES medium on a MEF monolayer in a T75 flask. Colonies were allowed to grow for 5 days prior to karyotyping. Karyotyping of 20 cells from the culture was performed at the Wisconsin State Laboratory of Hygiene.

D. Statistics

All experiments, with the exception of karyotyping, were repeated at least three times. Statistical analysis includes determination of mean, standard deviation, standard error of the mean, and a p-value using a one-paired one-tailed Student t-test, with p <0.05 being considered as significant.

Also it is envisioned that alkaline phosphatase activity can be used to verify the undifferentiated state of ES cells (Pera, M. F., et al., (2000) J Cell Sci 113 (Pt 1): 5-10). Furthermore, flow cytometric analysis using an anti-CD34 antibody and a fluorescent secondary antibody may be used to identify certain fractions of stem cell populations that are capable of differentiating (Kaufman, D. S., et al., (2001) PNAS 98: 10716-10721).

### Example 12

Optimization of ES Cell Preservation on a Substrate.

In order to optimize the ES cell preservation protocol cells were grown to approximately 1000-10,000 cell colonies on Matrigel™ or laminin in medium conditioned by MEF. Media was then added containing 35 mM trehalose 1 day prior to freezing. A thin (100 micron) layer of Matrigel™ was added over the colonies 1 day before freezing. Subsequently, the growth medium was removed and freezing medium was added (5-10% DMSO, 30-90% FBS). The cells were frozen at 1° C. per minute to −80° C. and stored in liquid Nitrogen. After storage, the cells were rapidly thawed in a 37° C. water bath. The freezing medium was aspirated and replaced with fresh ES cell growth medium. The media was changed daily and passaged when colony size was greater than 10,000 cells.

### Example 13

Preservation of Cells Grown on Matrigel™-Coated Microspheres.

In another embodiment, the invention provides a method for growing cells on microspheres to preserve them in an adherent manner, yet be able to pack them into cryovials so as to utilize current freezing equipment. One disadvantage of the above-described embodiments is that they require freezing plates, which can take up large freezer volume. In contrast, cells can be grown and preserved on ECM coated beads and stored in cryovials. Suitable beads used in accordance with the method of the invention are Cytodex™ microcarriers and have a diameter in the range of about 200 to 400 microns. Preferably the beads are 300 micron Matrigel™-coated microspheres.

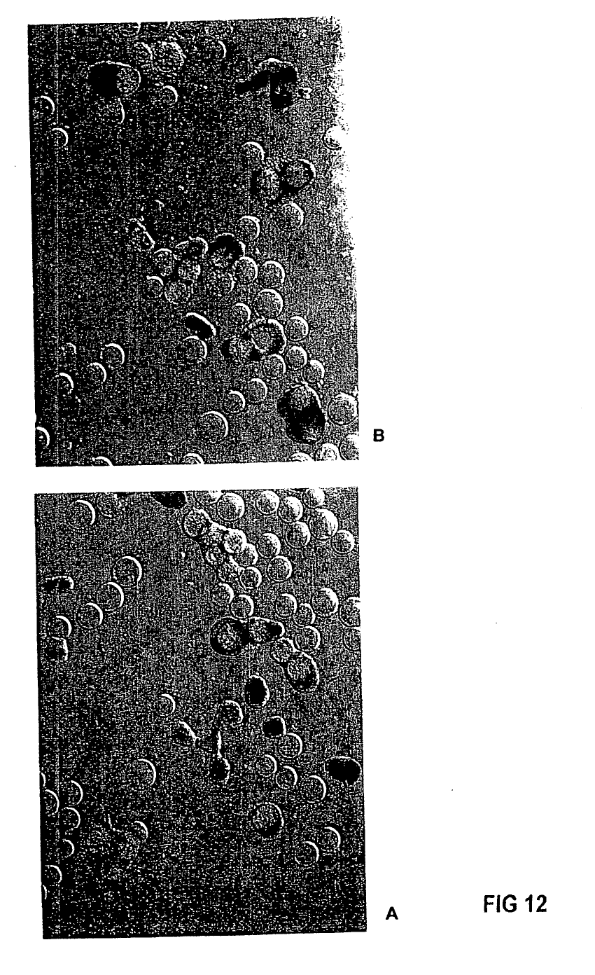

In a preferred embodiment, Cytodex 3 microcarriers (Amersham Biosciences) were coated with laminin (2 μg/cm²) or Matrigel™ (34 μg/cm²) overnight at 4° C. Microcarriers were vortexed during the coating to lessen clumping. After coating, microcarriers were washed with Ca²⁺/Mg²⁺ free PBS. HES colonies were detached by adding 1 ml of 1 mg/ml collagenase in DMEM/F12 to each well of the 6-well plate and incubating the plate at 37° C. for 5-10 min. Then the colonies were scraped off the plate and partially dissociated by gentle pipetting. Cells were washed twice and resuspended in CM/F+. Cells were then added to a monolayer of microcarriers in one well of a 24 well plate. Cells were incubated with microcarriers at 37° C. in a humidified incubator with 5% CO₂ for 24 hours to ensure attachment, as shown in FIG. 12. In referring to FIG. 12, it shows a 4× magnification of a day 7 culture on laminin-coated Cytodex 3 microcarriers. This figure shows a greater percentage of microcarriers covered with cells. Applicants note that clumping of microcarriers and cells is still an issue which will soon be overcome. After the attachment phase, the microcarriers and cells were transferred to an agitated vessel on an orbital shaker at 100 rpm. Cells were grown and maintained in CM/F+ media, which was changed daily. Also as described herein, the cells can be grown and maintained on any media (conditioned or unconditioned) that is capable of inhibiting differentiation.

Furthermore, it is envisioned that in practicing the methods of the invention an optimum freezing rate will be used, which is fast enough to minimize physical damage from ice crystal formation by forming vitrified water.

Results

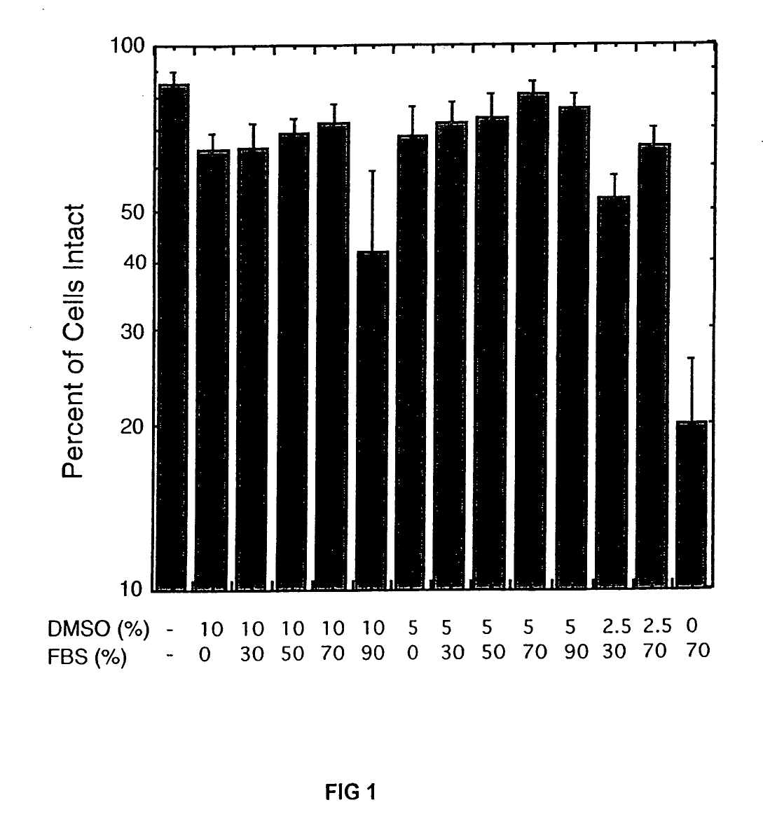

Standard HES cryopreservation methods consist of suspending colonies in cryopreservation media containing DMSO, FBS, and growth medium, followed by a slow rate of freezing to −70° C. then storage in liquid nitrogen (Thomson et al., 1998). As indicated earlier, survival rates of cells following these methods is poor, and cells that survive often differentiate (Reubinoff et al., 2001). Applicants varied the composition of the freezing medium to optimize cell viability following freezing and thawing (FIG. 1).

In referring to FIG. 1, it illustrates that DMSO is able to protect HES cells from membrane rupture during cryopreservation. Specifically, approximately 10⁶ HES cells were harvested from a Matrigel™-coated substrate as intact colonies and placed in freezing medium containing the indicated concentrations of DMSO and FBS. DMSO concentration varied from 0 to 10% and FBS concentration varied from 0 to 90%. The remainder of the freezing medium was CM/F+ medium. Colonies were removed from the plate and preserved as colonies, not as dispersed cells. Dispersion of cells results in virtually zero viability (data not shown).

Samples were frozen at approximately −1° C. per minute to −70° C. then stored in liquid nitrogen for 5 days. Following thawing and dispersion by trypsin, microscopic observation of freshly-thawed cells dyed with Trypan blue was performed to determine the number of intact cells; note, however, that a positive result in this simple assay does not necessarily mean that the cells are capable of growth. The number of cells that incorporate Trypan blue indicates the amount of damage to the cell membrane caused by the cryopreservation process. After harvest from the Matrigel™ substrate, over 80% of the HES cells exclude Trypan blue. The number of cells that exclude Trypan blue dropped only slightly to between 60% and 80% for most combinations of DMSO, FBS, and CM/F+ medium. Sample 1 (-DMSO, -FBS) indicates the fraction of cells excluding Trypan blue at the time of harvest from the Matrigel™-coated plate. Significantly more cellular damage occurred if no DMSO was present in the cryopreservation solution. From these results, it appears that a DMSO-FBS-CM/F+ mixture can protect the HES cells from extensive membrane damage during the freezing and thawing process. In reference to FIG. 1, it is noted that error bars represent SEM for at least 3 independent trials.

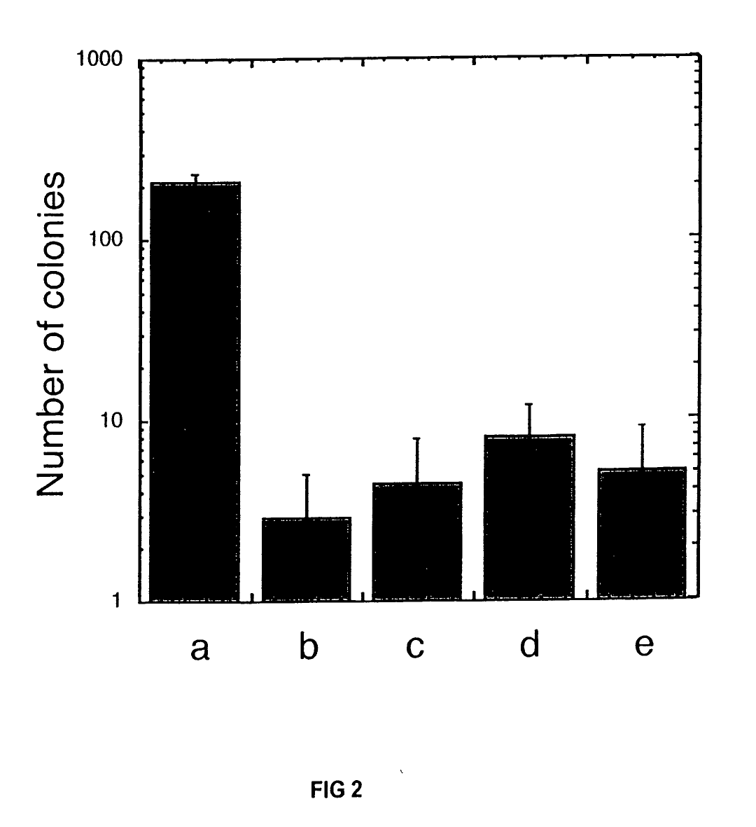

To determine how many of the colonies (frozen under the conditions described in FIG. 1) that permit adequate Trypan blue exclusion can actually reattach and resume growth, applicants rapidly thawed a cryopreserved cell sample containing 10⁶ cells, determined by counting on a hemacytometer, and plated the colonies on Matrigel™. In this experiment, colonies were frozen and replated as colonies. Cells were not dispersed at any time. After 1 to 2 weeks, the number of colonies growing on the plates was counted (FIG. 2). FIG. 2, illustrates few HES colonies cryopreserved in suspension attach and grow when replated on Matrigel™.

Specifically, in referring to FIG. 2, approximately 10⁶ HES cells were harvested from a Matrigel™ substrate as intact colonies and (a) replated on Matrigel™ or placed in (b) 5% DMSO 30% FBS, (c) 5% DMSO 70% FBS, (d) 10% DMSO 30% FBS, or (e) 10% DMSO 70% FBS and frozen as colonies at approximately −1° C. per minute to −70° C. Samples b-e were stored 5 days in liquid nitrogen and thawed colonies were replated on a Matrigel™-coated substrate. After 1-2 weeks of growth, the number of viable colonies on the plate was counted. It is noted that error bars represent SEM for at least 3 independent trials.

In relation to FIG. 2, approximately 40% of colonies directly passaged without freezing were able to reattach to the Matrigel™ substrate and grow. However, fewer than 2% of the colonies frozen and thawed in cryovials in a mixture of DMSO, FBS, and CM/F+ were able to attach to Matrigel™ and resume growth. Given that each colony contains on average approximately 2000 cells (determined by counting colonies, then dispersing and counting cells), and assuming that not every cell in the colony survived the cryopreservation process, the actual cell viability is likely well below 1%. Colony size was highly variable, however, with colony diameters varying by as much as a factor of three. Even though the number of cells excluding Trypan blue decreases by less than 25% when comparing control and frozen cells, the number of viable colonies drops by almost two orders of magnitude. Thus, a mechanism other than membrane permeation appears to be responsible for the low viability of HES cells following cryopreservation.

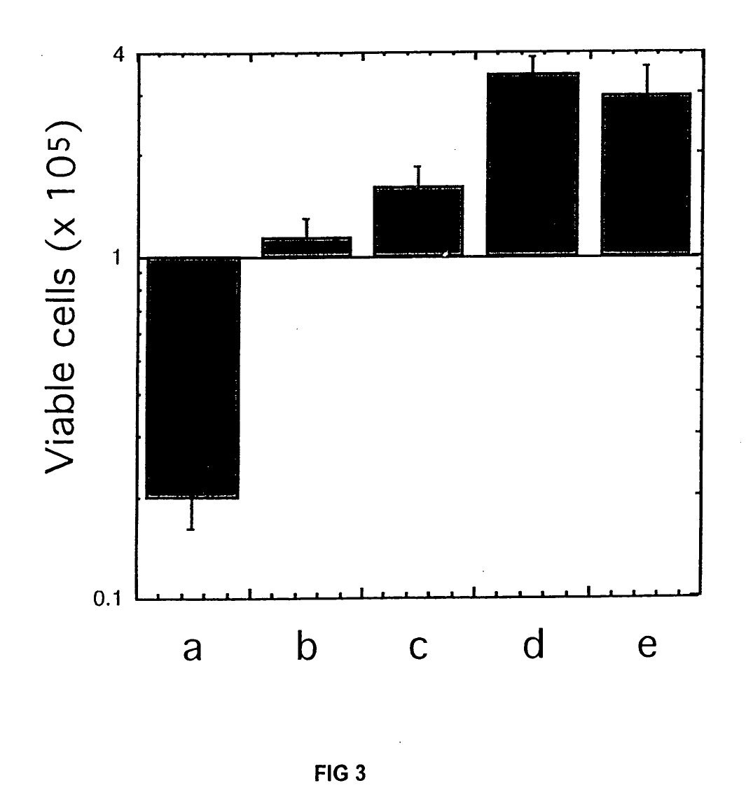

During passaging, the majority of colonies were unable to reattach (FIG. 2). This obviously poses problems during cryopreservation of colonies in suspension, since one expects damaged colonies to be less likely to attach to a Matrigel™ or MEF substrate than healthy colonies. To try to improve colony viability following cryopreservation, applicants developed a system to cryopreserve adherent HES cells. Approximately 10⁶ HES cells were cultured in each well of a 24 well plate. Cryopreservation of cells harvested from the wells and frozen in solution was compared with cryopreservation of cells on a Matrigel™-coated substrate and with cryopreservation of cells embedded in Matrigel™ (e.g., in a sandwich-like manner) for time periods ranging from 1 hr to 2 days (FIG. 3). FIG. 3 graphically illustrates HES colonies cryopreserved adherent to a Matrigel™ substrate exhibit higher viability upon thawing than HES colonies frozen in suspension.

Specifically, in referring to FIG. 3, approximately 10⁶ HES cells were cultured in each well of a Matrigel™-coated 24 well plate. Colonies in sample a were harvested from the plate as intact colonies and suspended in cryopreservation media containing 10% DMSO, 30% FBS, and 60% CM/F+. Samples b-e were preserved attached to the Matrigel™ layer in the same cryopreservation media. Sample b was preserved without an additional layer of Matrigel™ poured over the cells prior to freezing, c with a Matrigel™ layer poured 1 hr prior to freezing, d with a Matrigel™ layer poured 24 hr prior to freezing, and e with a Matrigel™ layer poured 48 hr prior to freezing. All samples were stored in liquid nitrogen and thawed after 3 days.

After thawing suspended cells on a Matrigel™-coated well and adherent cells in the wells in which they were frozen, colonies were allowed to recover (i.e., grown) for three days before measuring cell viability via MTT and Alamar blue assays. This recovery time is less than one doubling time following cryopreservation. As shown in FIG. 3, cells in colonies frozen while adherent were at least a factor of five more viable than cells in colonies frozen in suspension. Encapsulating colonies inside Matrigel™ for one or two days increased viability, compared to freezing adherent but unencapsulated colonies or colonies encapsulated for just one hour prior to freezing. It is noted that error bars represent SEM for at least four independent trials. Freezing medium containing FBS concentrations from about 30% to about 90% was equally effective in cryopreserving adherent HES colonies (not shown).

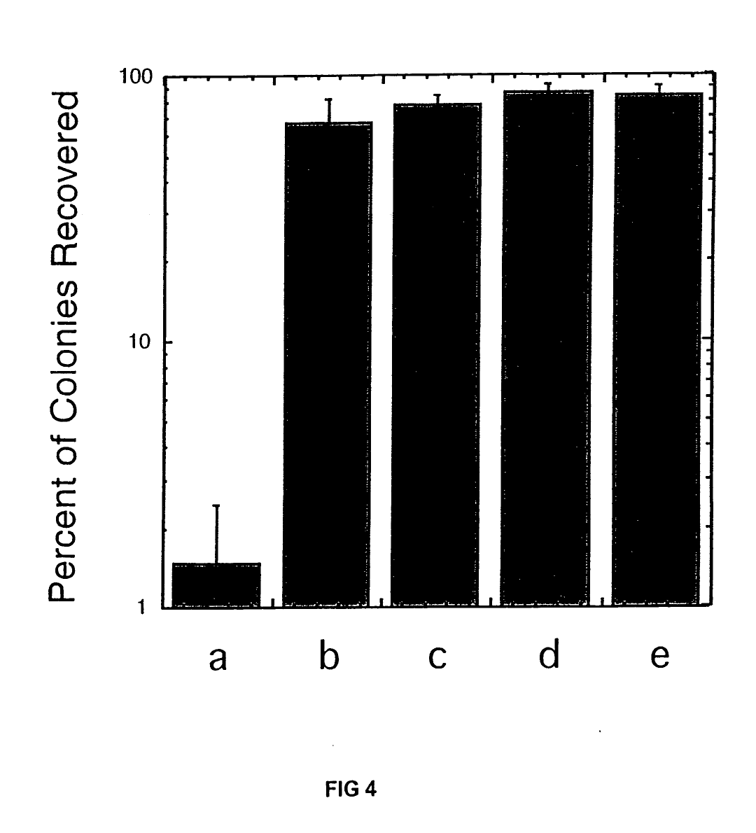

In addition to providing higher cell viability, cryopreservation of adherent colonies increases the number of colonies that are able to remain attached to the surface and eventually grow (i.e., higher recovery rate). FIG. 4 graphically illustrates cryopreservation of adherent HES colonies significantly increases colony recovery rate compared to cryopreservation of colonies in suspension. In referring to FIG. 4, approximately 10⁶ HES cells were cultured in the well of a Matrigel™-coated 24 well plate. Colonies in sample a were harvested from the plate as intact colonies (were not dispersed) and suspended in cryopreservation media containing 10% DMSO, 30% FBS, and 60% CM/F+. Samples b-e were preserved attached to the Matrigel™ layer in the same cryopreservation media. Sample b was preserved without an additional layer of Matrigel™ poured over the cells prior to freezing, c with a Matrigel™ layer poured 1 hr prior to freezing, d with a Matrigel™ layer poured 24 hr prior to freezing, and e with a Matrigel™ layer poured 48 hr prior to freezing. All samples were stored in liquid nitrogen and thawed after 3 days, then grown for 1-2 weeks. Colonies were then counted to determine colony recovery. Error bars represent SEM for at least four independent trials.

As shown in FIG. 4, fewer than 2% of colonies frozen in suspension were able to attach, while virtually all colonies encapsulated in Matrigel™ remained attached. Note that the cells frozen in suspension were maintained as colonies and were not dispersed. Encapsulating colonies in Matrigel™ prevented adherent colonies from detaching from the substrate and the length of time the colonies were frozen had little effect on the probability of detachment.

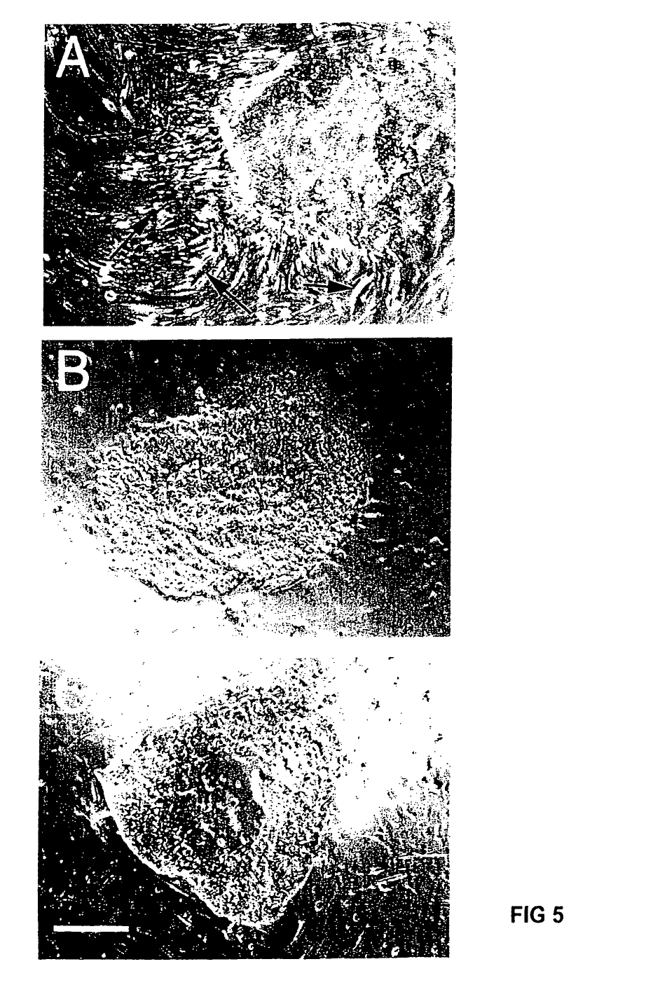

A common problem with freezing HES colonies in suspension is a large fraction of cells that do survive differentiate shortly after thawing. In FIG. 5A, a colony of HES cells preserved in suspension is observed after 5 days of growth post-thaw. This colony exhibits a high degree of differentiation, consisting of a core of undifferentiated ES cells surrounded by differentiated cells. In contrast, cryopreserving adherent HES colonies, either encapsulated in Matrigel™ or not, reduces differentiation (FIG. 5B, 5C). Note that colony morphology changes upon encapsulation in Matrigel™ (FIG. 5C).

In referring to FIG. 5, HES colonies were grown to an average of approximately 1000 cells each, determined by counting colonies then dispersing colonies and counting individual cells, on Matrigel™ and preserved as intact colonies in medium containing 10% DMSO, 30% FBS, 60% CM/F+in suspension (A), adherent to Matrigel™ without a top layer of Matrigel™ (B), and embedded in Matrigel™ for 24 hr prior to freezing (C). Samples were frozen at approximately −1° C. to −70° C. and stored in liquid nitrogen for 5 days prior to thawing. Colonies were allowed to grow 6 days then images were taken via phase contrast microscopy. It is noted that the arrows indicate differentiated cells and the scale bar is equal to 250 μm. It has also been observed that a culture of non-frozen HES cells embedded in Matrigel™ resulted in a similar morphology and lack of differentiation (not shown).

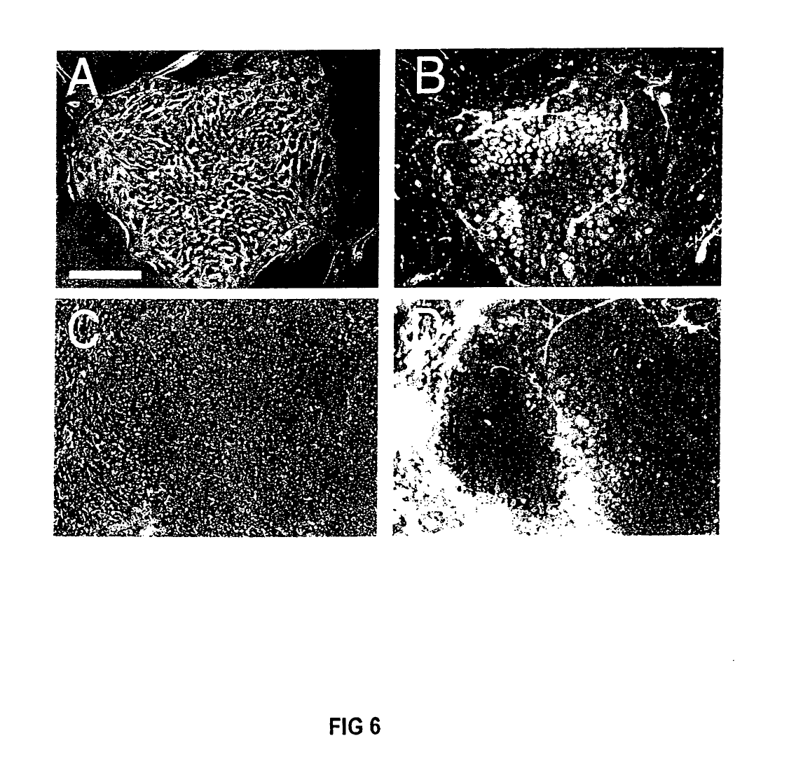

Anti-Oct4 immuno-staining of the colonies indicates that the colony body is composed of HES cells for colonies frozen adherent to or embedded in Matrigel™ (FIG. 6). In referring to FIG. 6, HES colonies were grown in 24 well plates on Matrigel™ in CM/F+ medium and preserved adherent to Matrigel™ in cryopreservation media containing 10% DMSO, 30% FBS, and 60% CM/F+ medium, with (A, B) or without (C, D) a layer of Matrigel™ poured over the colonies 24 hours prior to freezing. Samples were frozen at approximately −1° C. to −70° C. and stored in liquid nitrogen for 5 days prior to thawing. Subsequently, anti-Oct4 immunostaining was performed immediately following thawing (as shown in FIGS. 6A-D; Bar=50 μm).

In accordance with the invention, applicants note that the high degree of background in the Oct4 immunofluorescence is due to autofluorescence from dead cells in the colony. Furthermore, it was observed that cell shape and size, as well as colony morphology, change upon culture of colonies embedded in Matrigel™. Cells cryopreserved adherent to or embedded in Matrigel™ maintain Oct4 expression and normal HES morphology for greater than 20 passages, upon which applicants stopped culturing (not shown).

Applicant believe that enhanced survival of colonies attached to and embedded in Matrigel™ suggests that DMSO exposure has less of an effect on HES cell differentiation following cryopreservation than ECM signaling since cells exposed to DMSO do not differentiate if encapsulated in Matrigel™ throughout the freezing and thawing process. Together, the increases in number of adherent colonies and individual cell viability and inhibition of differentiation obtained by cryopreserving adherent HES cells can have significant effects on the time required to amplify cultures upon thawing. For example, 6±2 days are required to grow 10⁶ viable HES cells from 10⁵ cells cryopreserved encapsulated in Matrigel™, while generating the same number of viable cells from 10⁵ cells frozen in suspension requires 36±8 days.

Cryopreservation of HES cell colonies adhering to a MEF monolayer provides qualitatively similar increases in cell viability as seen on Matrigel™; virtually all colonies remain adherent and able to resume growth upon thawing and almost no differentiation is detected (data not shown). Pouring a Matrigel™ layer over HES colonies on MEF monolayers enhances viability. Applicants were not able to quantify cell viability, however, because the MEF viability interferes with the metabolic assays. MEF monolayer viability drops by approximately 50% during cryopreservation.

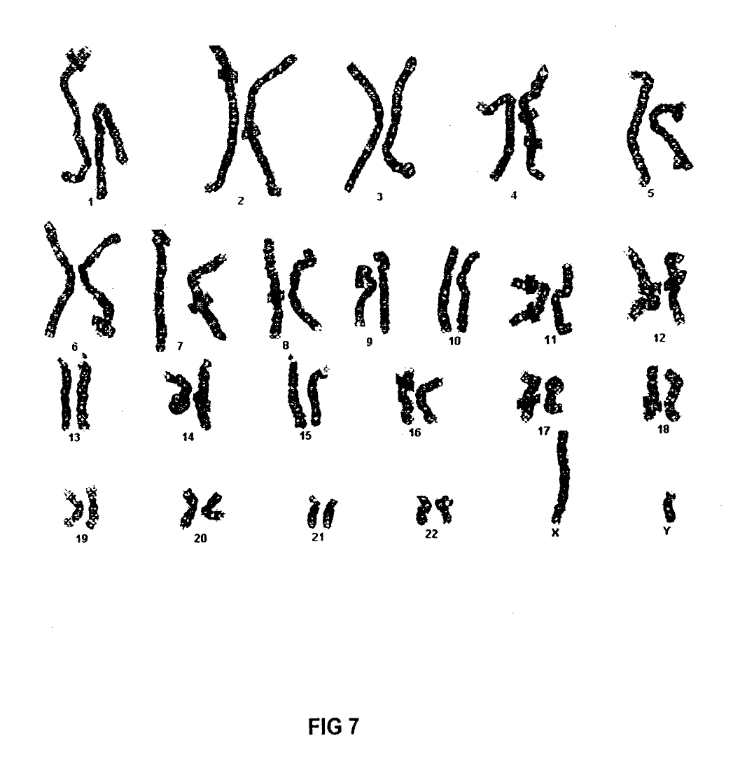

Furthermore, chromosomal changes, including gain of chromosome 17q and 12, have been observed in long-term culture of human embryonic stem cells, presumably due to a selective advantage for these aneuploidies (Draper et al., 2004). To determine if cryopreservation of adherent cells results in similar chromosomal changes applicants performed a karyotype analysis of HES cells frozen in colonies embedded in Matrigel™ for 24 hrs prior to freezing. As shown in FIG. 7, karyotype of thawed cells was normal male, although multiple freeze-thaw cycles might provide additional selection pressures that could result in abnormalities. Specifically, in referring to FIG. 7, HES colonies (H1, p29) were grown in 24 well plates on Matrigel™ in CM/F+ medium and preserved as intact colonies in cryopreservation media containing 10% DMSO, 30% FBS, 60% CM/F+ adherent to Matrigel™. The colonies were embeddded in a Matrigel™ 24 hr prior to freezing. Samples were frozen at approximately −1° C. to −70° C. and stored in liquid nitrogen for 5 days prior to thawing. Cells were harvested from the Matrigel™ by treatment with 1 mg/ml collagenase and transferred to an irradiated MEF monolayer in a T75 flask. Colonies were allowed to grow 5 days and karyotype analysis was performed on 20 cells, which were observed to have a normal male karyotype.

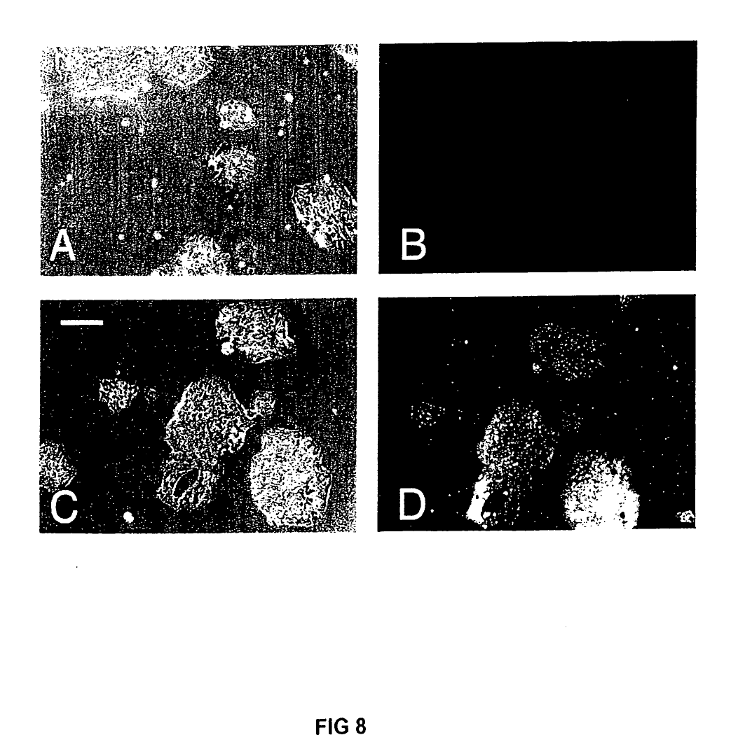

Also, as mentioned hereinabove, because DMSO is a cytotoxic, penetrating cryoprotectant thought to contribute to differentiation of HES cells, applicants investigated whether trehalose could replace DMSO or enhance viability of cells in the presence of DMSO. First, applicants attempted to determine effective loading conditions for trehalose into ES cells and colonies. Trehalose, in the past, has been typically loaded into mammalian cells via fluid-phase endocytosis (Wolkers et al., 2001). Following Wolkers et al., applicants used a tracer dye, Lucifer Yellow (m.w.=450 Da), to simulate trehalose (m.w.=342 Da) loading via fluid phase endocytosis. Applicants found that cell exposure to growth medium containing trehalose at concentrations greater than 35 mM caused toxicity (not shown). By exposing cells to 35 mM Lucifer Yellow for varying time periods applicants found maximum loading occurs at 24 hrs. FIG. 8 shows that Lucifer Yellow is evenly distributed throughout a HES cell colony exposed to a 35 mM solution of the dye. In referring to FIG. 8, HES colonies were grown in CM/F+ medium containing 0 mM lucifer yellow (A, B) or 35 mM lucifer yellow (C, D) for three hr. Colonies were rinsed with CM/F+ medium 5 times and phase (A, C) and epifluorescence images taken (B, D). In FIG. 8, the scale bar is equal to 150 μm. On the basis of these results applicants expected trehalose to load into cells throughout the colony after a 24 hr exposure.

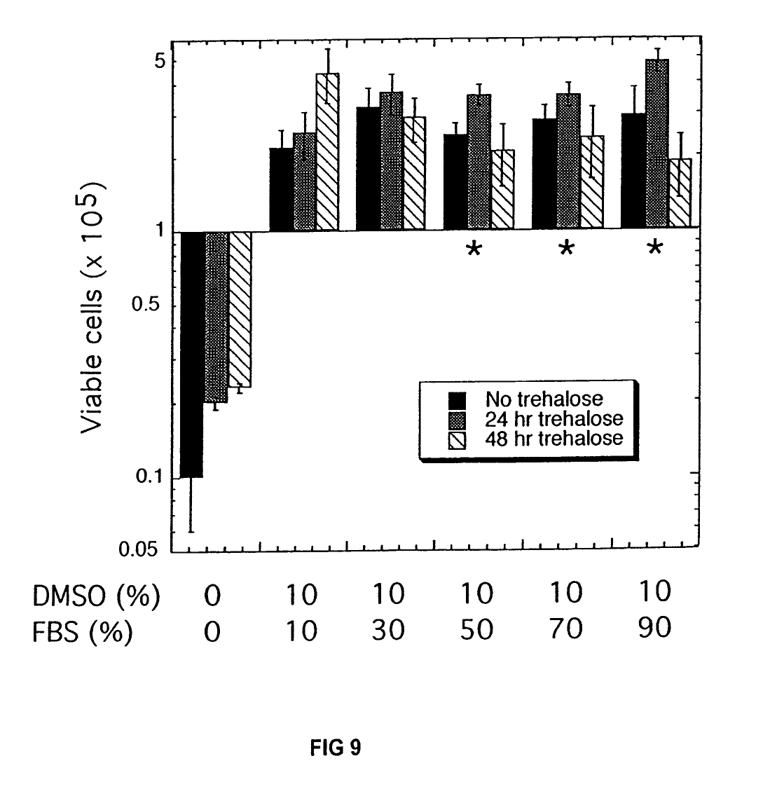

To assess whether trehalose can protect cryopreserved HES cells, applicants loaded HES cells with trehalose for 24 or 48 hours prior to freezing. As shown in FIG. 9, approximately 10⁶ HES cells were cultured in each well of a Matrigel™-coated 24 well plate. Colonies were loaded with trehalose by incubation in CM/F+ medium containing 35 mM trehalose for 24 or 48 hr prior to preservation. Trehalose loading medium was replaced with freezing medium containing the indicated percentages of DMSO and FBS, then adherent colonies, without a layer of Matrigel™ poured over them, were frozen at approximately −1° C. per minute to −70° C. Samples were stored in liquid nitrogen for 5 days then thawed and grown for 5 days prior to determining cell concentration via MTT or alamar blue assays. * indicates loading with trehalose for 1 day is significantly better than no trehalose (p<0.05). Error bars represent SEM of at least 4 independent trials.

Applicants observed that in the absence of DMSO, trehalose provides no protection during freezing and thawing. At 10% DMSO and FBS concentrations less than 50%, trehalose provides no additional protection. However, at 10% DMSO and FBS concentrations above 50%, loading cells with trehalose for 24 hours provides a 25% increase in viability compared to DMSO alone (p<0.05). Loading cells with trehalose for 48 hours generally results in lower viability than loading for 24 hours, or not loading at all, perhaps due to osmotic stresses on the cells during the loading process.

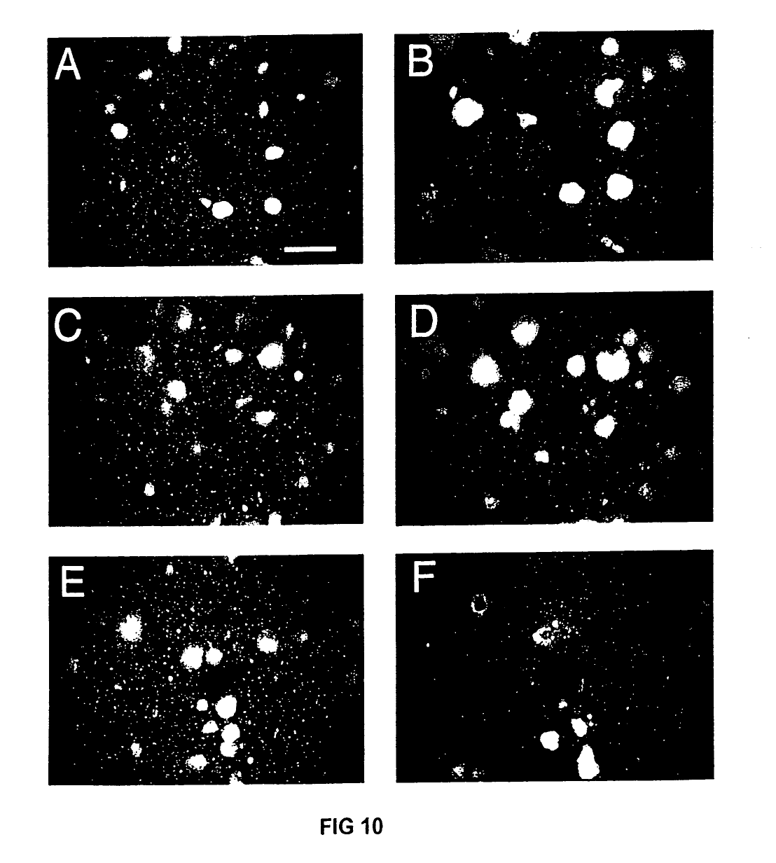

Loading cells with 35 mM trehalose for 24 hours prior to freezing, and then freezing in medium containing DMSO results in an increase in the number of adherent colonies following thawing; virtually all colonies remain attached to the surface and can grow (FIG. 10). Specifically, in referring to FIG. 10, HES colonies were cultured in each well of a Matrigel™-coated 24 well plate. Colonies were loaded with trehalose by incubation in CM/F+ medium containing no trehalose (A, B) or 35 mM trehalose for 24 (C, D) or 48 hr (E, F) prior to preservation. Trehalose loading medium was replaced with freezing medium containing the indicated percentages of DMSO and FBS, then adherent colonies, without a layer of Matrigel poured over them, were frozen at approximately −1° C. per minute to −70° C. Samples were stored in liquid nitrogen for 5 days then thawed and grown for 5 days. Images of the same field were acquired by phase contrast microscopy just prior to freezing (A, C, E) and 5 days after thawing (B, D, F). Scale bar=1 mm. Applicants observed that after freezing and thawing cells loaded with 35 mM trehalose for 24 hours, 82%±5% of colonies were recovered, similar to results obtained in the absence of trehalose (FIG. 4). However, loading with trehalose for 48 hours causes many colonies to detach from the Matrigel™ substrate during freezing and thawing; here only 48%±10% of colonies were recovered.

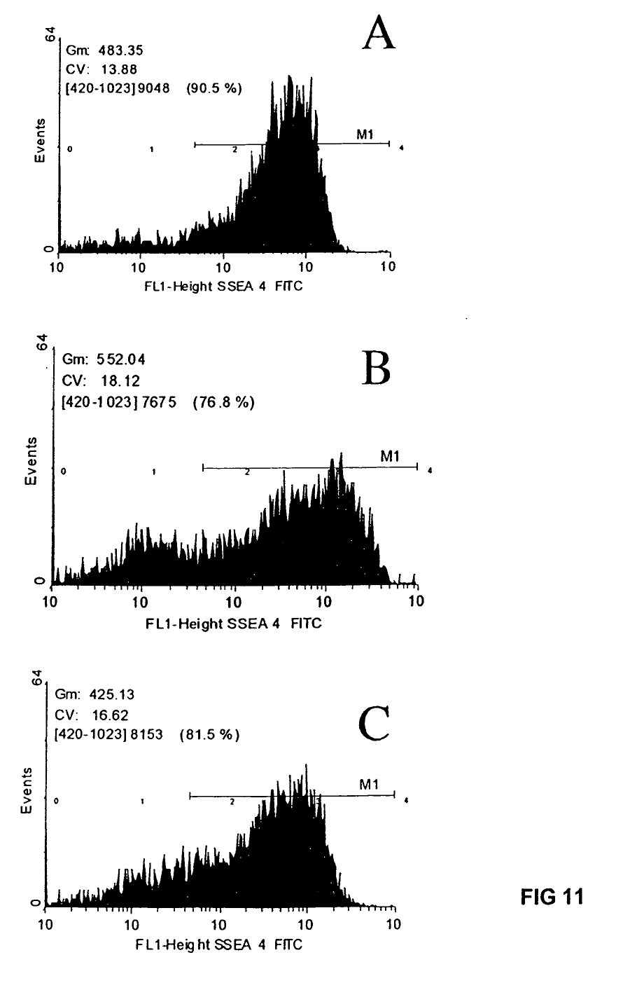

Furthermore, in addition to retention of OCT4 expression (see FIG. 6), applicants observed that when HES colonies were frozen in suspension in cryovials, adherent to Matrigel™, embedded in Matrigel™, and loaded with trehalose were able to maintain SSEA4 expression after recovery (FIG. 11, Table 1). Specifically, in referring to FIG. 11, HES colonies were cultured in each well of a Matrigel™-coated 24 well plate. Sample A was continuously cultured prior to flow cytometry. Sample B was preserved in suspension without trehalose loading and Sample C was preserved adherent to Matrigel™ following incubation in CM/F+ medium containing 35 mM trehalose for 24 hr prior to cryopreservation, as described in the examples above. Samples B and C were frozen at −1° C./min medium containing 10% DMSO, 30% FBS, and 60% CM/F+ medium and stored in liquid nitrogen for 5 days prior to thawing. After thawing, cells were cultured in CM/F+ medium for 7 days prior to SSEA4 expression analysis. The gated region for all samples was determined from the positive control (Sample A). Then, cells were stained for SSEA4 and number of SSEA4+ cells determined by comparing to positive controls generated by staining HES cells in continuous culture. A negative control was generated using mouse IgG3. All cryopreserved samples exhibit similar levels of SSEA4+ staining, slightly lower than the level of SSEA4+ staining found in the positive control. These results are in contrast to the observations of phase contrast and Oct4 immunocytochemistry that indicate a much higher level of differentiation in colonies preserved in suspension. Part of this discrepancy may be due to the longer time allowed for recovery and growth prior to flow cytometry. Also, it is noted that positive expression is determined somewhat arbitrarily based on expression in HES cells growing in culture.

Results for additional preservation protocols are summarized in Table 1. Specifically, Table 1 summarizes SSEA4 expression on cryopreserved HES cells determined by flow cytometry.

TABLE 1

| | Percent of |
|---|---|
| Sample Preservation Method | cells gated |
| Negative control (not preserved, labeled with mouse IgG) | 1.3 |
| Positive control (continuous cultured HES cells, not | 90.5 |
| preserved) | |
| Suspension | 76.8 |
| Attached to Matrigel ™ | 76.8 |
| Embedded in Matrigel ™ for 1 hr | 82.8 |
| Embedded in Matrigel ™ for 2 days | 77.2 |
| Embedded in Matrigel ™ for 2 days | 81.0 |
| Attached to Matrigel ™, loaded with 35 mM trehalose for 1 | 81.5 |
| day | |

All cells were preserved in 10% DMSO, 30% FBS, 60% CM/F+ medium

Discussion

One of the main challenges facing stem cell research and translation of research progress to clinical settings is the ability to efficiently and effectively grow and preserve HES cell lines. Standard cryopreservation methods using slow freezing of cells in suspension kill the vast majority of HES cells (FIG. 2) (Reubinoff et al., 2001). While Reubinoff et al. (2001) report that 16% of colonies frozen using standard methods can be recovered as small HES colonies with high levels of differentiation, applicants recovered approximately 2% using similar methods (FIG. 2). This difference in recovery may be due to differences in cell lines, freezing and thawing protocols, or growth substrate (MEF cells vs. Matrigel™). Nevertheless, recovery of HES cells cryopreserved in suspension is low and purification of cultures is time-consuming. While HES cells are immortal and thawed samples can be grown until the required number of cells are obtained (Amit et al., 2000), this low viability causes numerous problems. First, the time between thawing a sample and performing experiments or clinical use slows the pace of research and therapy considerably. Inter-laboratory transport of ES cell lines is often difficult and ineffective. Cell properties may change as passage number increases during expansion. The low viability rate also induces selection pressures during freeze-thaw cycles; mutations that increase cell survival will be strongly selected, and these mutations may affect immortality or pluripotency of the cell lines. Thus, the above-described embodiments of the invention demonstrate that cryopreservation of encapsulated HES cells offers better cellular viability, higher colony recovery, and less differentiation than the slow freezing techniques most commonly used to preserve HES colonies.

Applicants believe that preservation of adherent HES colonies increases viability through a number of potential mechanisms, suggesting that membrane preservation may not be the limiting factor in designing an appropriate cryopreservation strategy for HES cells. Indeed, targeting other factors such as extracellular matrix (ECM) signaling, stress minimization, or apoptosis inhibition might be more effective for enhancing viability and reducing differentiation of HES cells cryopreserved in an adherent state. The fact that cells embedded in Matrigel™ for a day survive freezing to a greater extent than cells embedded in Matrigel™ for only an hour suggests that an increased level of ECM signaling may prepare the HES cells for the stresses associated with freezing and thawing. Applicants anticipate that cryopreservation of adherent HES colonies confers increases in stress-response signaling and anti-apoptotic activity. For example, Caspase transcription increases in fibroblasts that survive cryopreservation, and addition of Caspase I Inhibitor V to the cryopreservation media increases cell viability upon thawing (Baust et al., 2000); HSP up-regulation was more pronounced in cells preserved in 3-D than cells preserved in suspension. Likewise, p38 mitogen activated protein kinase levels and growth factor transcription were higher in thawed 3-D cultures than in cells frozen in suspension (Liu et al., 2000).

Parameters other than those investigated in this study may also impact HES viability and differentiation during recovery. For example, colony size likely affects survival since dispersed cells do not form colonies. Maintaining appropriate cell-cell contacts as well as cell-substrate attachment may be critical for optimizing HES cell cryopreservation. Also, adjusting freezing rate and thawing rate may affect intracellular and extracellular ice formation. Studies to optimize these parameters and to investigate the effects on long-term storage are underway.

HES colony vitrification in open pulled straws is another preservation option that has been reported to be superior to slow freezing in suspension (Reubinoffet al., 2001). Vitrification is a more rapid, simpler protocol than preservation of adherent cells or cells in suspension for small number of colonies, but heat transfer limitations make it difficult to scale up for larger samples. During vitrification, colonies must be very small (100-200 cells) and only a few colonies can be stored per straw. While vitrification increases HES cell viability compared to slow freezing, it also increases spontaneous differentiation. Cryopreservation of adherent HES cells, in contrast, decreases differentiation, perhaps due to maintenance of anti-differentiative signals from the Matrigel™.

DMSO is cytotoxic and thought to contribute to the differentiation of HES cells upon thawing. Therefore, if cryopreservation media can be supplemented with other less toxic protectants, DMSO concentration could be lowered or eliminated altogether. Trehalose is an attractive candidate since it has been effective in mammalian cell stabilization at low temperatures and water contents and appears to aid cell viability by different mechanisms than DMSO (Crowe et al., 2001; Sum and de Pablo, 2003). For example, trehalose addition to cryopreservation media containing DMSO and FBS increases the viability of hematopoeitic precursor cells by 7-20% and improves membrane integrity in cryopreserved fetal skin (Erdag et al., 2002). Applicants data has been able to show that trehalose can have beneficial effects during cryopreservation of HES cells in the presence of DMSO and at high FBS concentrations. The major drawback with using trehalose as a cryoprotectant is loading the disaccharide into cells. The use of Lucifer yellow to optimize trehalose loading is problematic in that molecular features other than size, such as charge or chemistry, may be important in trehalose loading. However, fluid phase endocytosis has been demonstrated to be a main mechanism of trehalose loading in human platelets (Wolkers et al., 2003). Further work must be performed to directly measure optimum intracellular trehalose concentrations, determine if specific mechanisms of trehalose uptake are important in HES cells, and develop more rapid mechanisms of loading to minimize osmotic damage to the cells. α-hemolysin, a pore-forming protein from Staphylococcus aureus has been introduced into mammalian cells to permit trehalose loading (Chen et al., 2001). However, this approach requires genetic modification of the cell line.

Furthermore, as noted in the Examples above, cryopreserving adherent cells, would require redesign of cell storage facilities, and would increase storage space necessary to preserve the same number of cells. However, methodologies such as preservation on microcarriers or in microscale gel particles might provide the advantages of freezing adherent cells at higher densities than are possible on flat surfaces.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of cryopreserving pluripotent stem cells, comprising the steps of:
a) growing the cells on a bottom layer of solid support matrix;
b) adding a top layer of solid support matrix over the cells, such that cells are maintained between the two layers of matrix forming a matrix-cell-matrix composition;
c) adding an effective amount of a cryopreservation medium over the matrix-cell-matrix composition; and
d) cooling the matrix-cell-matrix composition to a temperature sufficient to cryopreserve the cells.

2. The method of claim 1 further comprising thawing of the cryopreserved cells, such that the cells exhibit increased cell recovery, enhanced cell viability and decreased differentiation.

3. The method of claim 1 wherein the cells are mammalian cells.

4. The method of claim 1 wherein the cells are human embryonic stem cells.

5. The method of claim 1 wherein the cells are grown to about 1000 to 10,000 cell colonies.

6. The method of claim 1 wherein the solid support matrix is either porous or non-porous.

7. The method of claim 6 wherein the porous solid support matrix comprises Matrigel™ conditioned or unconditioned medium.

8. The method of claim 6 wherein the non-porous solid support matrix comprises polystyrene coated with extracellular matrix proteins.

9. The method of claim 1 wherein the bottom layer of solid support matrix comprises matrix-coated beads.

10. The method of claim 9 wherein the beads are coated with Matrigel™ or laminin.

11. The method of claim 1 wherein the cryopreservation medium comprises an effective amount of a carbohydrate-based medium followed by the addition of a freezing medium.

12. The method of claim 11 wherein the carbohydrate-based medium is poured over the matrix-cell-matrix composition about 2 to about 30 hours prior to the addition of the freezing medium.

13. The method of claim 11 wherein the carbohydrate is trehalose.

14. The method of claim 11 wherein the freezing medium comprises 10% DMSO, 30% FBS and 60% HES medium.

15. The method of claim 1 wherein the cooling temperature is about −70° C. to −195° C.

16. A matrix-cell-matrix composition comprising:
a) a bottom layer of solid support matrix;
b) embryonic stem cells grown on the bottom layer of matrix; and
c) a top layer of solid support matrix poured over the cells, such that cells are maintained between the two layers of matrix.

17. A method of cryopreserving embryonic stem cells, comprising the steps of:
a) growing the cells on a bottom layer of solid support matrix, such that the cells adhere to the matrix;
b) adding an effective amount of a cryopreservation media over the matrix adherent cells; and
c) cooling the matrix adherent cells to a temperature sufficient to cryopreserve the cells.

18. The method of claim 17 wherein the bottom layer of solid support matrix is either porous or non-porous.

19. The method of claim 18 wherein the porous solid support matrix comprises Matrigel™ in conditioned or unconditioned medium.

20. The method of claim 18 wherein the non-porous solid support matrix comprises polystyrene coated with extracellular matrix proteins.

21. The method of claim 17 wherein the bottom layer comprises matrix-coated beads.

22. The method of claim 21 wherein the beads are coated with Matrigel™ or laminin.

23. The method of claim 17 wherein the cryopreservation media comprises an effective amount of a carbohydrate-based medium followed by the addition of a freezing medium.

24. The method of claim 23 wherein the carbohydrate-based medium is poured over the adherent cells about 18 to about 30 hours prior to the addition of the freezing medium.

25. The method of claim 23 wherein the carbohydrate is trehalose.

26. The method of claim 23 wherein the freezing medium comprises 10% DMSO, 30% FBS and 60% HES medium.

27. The method of claim 17 further comprising thawing of the cryopreserved cells, such that the cells exhibit increased cell recovery, enhanced cell viability and decreased differentiation.

28. A method of enhancing viability and recovery and decreasing differentiation during cryopreservation of embryonic stem cells comprising the steps of:
a) growing the cells on a bottom layer of solid support matrix, such that the cells adhere to the matrix;
b) adding a top layer of solid support matrix over the cells, such that cells are maintained between the two layers of matrix forming a matrix-cell-matrix composition;
c) adding an effective amount of a cryopreservation medium over the matrix-cell-matrix composition;
d) cooling the matrix-cell-matrix composition to a temperature sufficient to cryopreserve the cells; and
e) thawing the composition, such that the cells have enhanced cell viability and decreased differentiation as compared to cells not having been cryopreserved within a matrix-cell-matrix composition.

29. The method of claim 28 wherein the solid support matrix is porous or non-porous.

30. The method of claim 29 wherein the porous solid support matrix comprises Matrigel™ in conditioned or unconditioned medium.

31. The method of claim 29 wherein the non-porous solid support matrix comprises polystyrene coated with extracellular matrix proteins.

32. A method of enhancing viability and recovery and decreasing differentiation during cryopreservation of embryonic stem cells comprising the steps of:
a) growing the cells on a bottom layer of solid support matrix, such that the cells adhere to the matrix;
b) adding an effective amount of a cryopreservation medium over the matrix adherent cells;
c) cooling the matrix adherent cells to a temperature sufficient to cryopreserve the cells; and
d) thawing the cells, wherein the thawed cells have enhanced cell viability, recovery and decreased differentiation as compared non-matrix adherent cells.

33. The method of claim 32 wherein the solid support matrix is porous or non-porous.

34. The method of claim 33 wherein the bottom layer of matrix comprises matrix-coated beads.
